(19) United States
(12) Patent Application Publication
(10) Pub. No.: US 2017/0045532 A1
(43) Pub. Date: Feb. 16, 2017

(54) MATERIALS AND METHODS FOR DIAGNOSIS OF PERI-IMPLANT BONE AND JOINT INFECTIONS USING PROPHENOLOXIDASE PATHWAY

(71) Applicants: Javad PARVIZI, Gladwyne, PA (US); Pouya ALIJANIPOUR, Philadelphia, PA (US)

(72) Inventors: Javad PARVIZI, Gladwyne, PA (US); Pouya ALIJANIPOUR, Philadelphia, PA (US)

(21) Appl. No.: 15/306,005

(22) PCT Filed: Apr. 17, 2015
(86) PCT No.: PCT/US2015/026279

§ 371 Date: Oct. 21, 2016

Related U.S. Application Data
(60) Provisional application No. 61/982,531, filed on Apr. 22, 2014.

(51) Int. Cl. G01N 33/68; C12Q 1/04; C12Q 1/26

(52) U.S. Cl. CPC G01N 33/6887; C12Q 1/26; C12Q 1/04; G01N 2800/245

(57) ABSTRACT

Diagnostic assays and related systems, test samples and methods are described for detection of infection following implantation of a prosthetic material or device on and/or into bones and joints. A combination of at least two biomarkers is used, one specific to the pathogenic microbe and the other specific to the destruction of peri-implant tissues. The biomarkers are detectable in the peripheral blood, urine, fluid or tissue samples drawn from a peri-implant environment. They can also be detected with a blood or urine test that is highly sensitive for detecting specific structural microbial molecules and peri-implant tissue components.

### BACKGROUND OF THE INVENTION

Field of the Invention

The present invention has to do with the detection of infection following implantation of a prosthetic material or device on and/or into bones and joints. The invention has both human and veterinary applications.

The Related Art

There is a risk of infection following implantation of any prosthetic material or device in a physiologically sterile site or organ such as bone and joints. Any kind of internal or external orthopedic implants, including but not limited to breast implants, prosthetic heart values, neurosurgical implants (such as vascular clips, shunt tubes and vascular stents) are just a few examples of prosthetic materials and devices that can be the subject of this invention. Diagnosis of infection following surgery can be difficult and in many cases the infection cannot be detected at an early stage. Early stage detection is important because it greatly enhances successful surgical outcomes.

Each infectious process is the conclusion of interaction between a virulent pathogen on one side and the host immune system on the other side. The pathogen causes structural damage to the host tissues and in cases with bone and joint infection, the pathogenesis of infection is associated with damage to peri-implant tissues cells as well as disruption of the extracellular matrix in those tissues. On the other hand, a sufficiently competent immune system will elicit an inflammatory reaction in response to the presence of the microbes which can also cause damage to host tissues as a secondary adverse effect. Therefore, during a peri-implant infection, structural molecules or components specific to microbial cells or host tissues (in this invention the host tissues include bone, cartilage, muscle, ligament, tendon, articular capsule, etcetera) are released into the peri-implant environment and a portion of these molecules will be absorbed into the blood circulation and from blood into the urine.

### SUMMARY OF THE INVENTION

This invention is based on the idea that a combination of at least two biomarkers, (one specific to the pathogenic microbe and the other specific to destruction of peri-implant tissues) that are detectable in the peripheral blood can be used for diagnosis of peri-implant infections. Any fluid or tissue samples drawn from a peri-implant environment (such as joint aspirate, tissue aspirate, tissue biopsy, etcetera) can be tested for the dual biomarker concept. Moreover, a blood or urine test that is highly sensitive for detecting specific structural microbial molecules and peri-implant tissue components can be convenient and accessible sources for diagnosing peri-implant infection of the bone and joints.

Some benefits of the present invention are as follows:

1. Prophenoloxidase (PPO) pathway can detect presence of peptidoglycan (PG, found in the bacterial cell wall) and betaglucan (BG, found in fungi) in the blood, urine, prosthetic membrane (biofilm) and periprosthetic tissue, including articular capsular, periprosthetic fibrotic tissue, fascia, ligaments, tendons, muscles and even subcutaneous tissue depending on the extent of the infection. Moreover, this phenomenon can be used to detect PG or BG in the tissue material formed at the interface of bone-implant, bone-cement and cement-implant.

2. Any biomarker of destruction of peri-implant tissues such as bone, cartilage, synovium, muscle, fat and other periparticular tissues can be measured in the blood, urine and periprosthetic fluid as indicators of ongoing damage to these tissues due to peri-implant infections. These biomarkers are catabolized or non-modified components of the damaged tissue cells or extracellular matrix and are released by the interaction of the pathogens and/or host immune system with the affected host tissue. Such markers can be identified and measured via antibody-based immunoassay methods (such as enzyme-linked immunosorbent assay or ELISA, radioimmunoassay or RIA, or lateral flow immunoassay) or other non-ELISA based methods (such as chromatography-based, mass-spectrometry-based, gel-electrophoresis-based, western blot, microarrays, metabolemic, lipidomic and proteomic methods) and include but are not limited to biomarkers of bone metabolism, degradation or remodeling, Biomarkers related to collagen metabolism, Biomarkers related to aggrecan metabolism, Biomarkers related to other non-collagenous proteins, Osteoblast-osteoclast regulating factors (Regulatory molecules of osteoblasts and osteoclasts and Biomarkers related to other metabolic processes of bone and joint tissues.

3. The “dual-marker” concept is considered as a combination of any form of diagnostic tests devised based on the above concepts. PPO pathway can be applied for several different specimens (such as blood, urine, peri-implant fluid or tissue samples) to measure the levels of PG or BG. The biomarkers of host tissue destruction can be measured in blood, urine and peri-implant fluid. One or any combination of the above mentioned biomarkers can be utilized in combination with PPO pathway-based test as a diagnostic assay according to the “dual-marker” concept.

4. In patients with persistent surgical wound drainage after primary or revision arthroplasty or other procedures (spine, trauma, etc), PPO pathway can be used to monitor the release of PG or BG into blood or urine. Positivity of dual-marker concept (persistently elevated or increasing levels of PG or BG associated with persistently elevated or increasing levels of biomarkers of damage to peri-implant tissues) can demonstrate early peri-implant postoperative infection of bone and joints.

5. In patients with failed symptomatic implants and suspicion of peri-implant infection of bone and joint, any diagnostic test based on the “dual-marker” concept can be used as a screening tool to measure the level of PG, BG and biomarkers of tissue damage. Detection of PG, BG and biomarkers of tissue damage in the serum can be indicative of peri-implant infection of bone and joint due to continuous release of PG, BG and biomarkers of tissue damage from infected peri-implant tissue and material into the blood.

6. The “dual-maker” concept can be used as a point-of-care test during surgical intervention in cases with failed implants, in whom suspicion of infection exists yet it has not been proven by the available microbiological and non-microbiological tests. Elevated levels of PG or BG in the intraoperative specimens (periprosthetic fluid or tissue samples) can confirm the role of infection in implant failure. This can have a critical impact on the surgeon's decision making during the operation regarding surgical plan and definitive postoperative surgical and medical treatment strategy.

7. The “dual-marker” concept can be used to follow the evolution of peri-implant infection and to determine whether surgical and medical treatment of peri-implant infection has been successful or failed.

8. In patients with peri-implant infection of the joints who undergo the first stage of a two-stage exchange arthroplasty (i.e. removal of the implant and placement of an antibiotic spacer), the “dual-marker” concept can be used to follow the evolution of infection and determine the optimal time for the second stage of this procedure (i.e. removal of the antibiotic spacer and implantation of new definitive prosthesis).

9. In patients with peri-implant infection of the joints who undergo second stage of two-stage exchange arthroplasty, the “dual-marker” concept can be utilized to measure PG, BG and biomarkers of peri-implant tissue destruction in blood and urine in a serial manner and as a follow up test to confirm the success of the treatment of NI.

The following lists various biomarkers.

Biomarkers of Bone Metabolism, Degradation or Remodeling
Bone isoenzyme of alkaline phosphatase (BALP)
Cathepsin K (osteoclastic enzyme)
Fibroblast growth factor-3 (FGF-3)
Midfragments of osteocalcin (MidOC)
Osteocalcin
Osteopontin
Osteoprotegerin
Osteoglycin
Periostin (POSTN)
Tartrate resistant acid phosphatase (TRACP)

Biomarkers Related to Collagen Metabolism
Alpha-helical region of type II collagen (Coll2-1) and its nitrated form (Coll2-1 NO2)
Aminoterminal propeptide of collagen type I (Procollagen type I N-terminal propeptide [PINP])
Beta-Carboxy-terminal crosslinked telopeptide of type I collagen (CTX-I, CTX-I alpha, CTX-I beta)
Carboxy-terminal crosslinked telopeptide of type I collagen (ICTP)
C-terminal telopeptide of collagen type I (s-βCTX)
C-terminal telopeptide of collagen type II (CTX-II)
Collagen type II-specific neoepitope (C2M)
Hydroxyproline
Matrix-Metalloproteases (MMP)-generated type I collagen fragment (CTX-MMP)
N-terminal telopeptide of type I collagen (NTX-I)
Procollagen type-1 Carboxytermina-propeptide (PICP)
Pyridinoline, Pyridinium Crosslinks, Deoxypyridinoline, Glc-Gal-PY
Type II collagen α-chains collagenase neoepitope (α-CTX-II)
Type II collagen cleavage product (C2C)
Type II collagen propeptides (PIINP, PIIANP, PIIBNP, PIICP, CPII)
Types I and II collagen cleavage neoepitope (C1,C2)

Biomarkers Related to Aggrecan Metabolism
Chondroitin sulfate and monoclonal antibody 3B3(−)
Core protein fragments (aggrecan neoepitopes such as CS846, ARGS and FFGV fragments)
Keratan sulfate

Biomarkers Related to Other Non-Collagenous Proteins
Cartilage oligomeric matrix proteins (COMP and its deamidated form D-COMP)
Cellular inhibitor of apoptosis protein (cIAP)
Fibulin (peptides of fibulin 3, Fib3-1, Fib3-2)
Follistatin-like protein 1 (FSTL-1)
Hyaluronan (hyaluronic acid)
Matrix metalloproteinases (MMP-1, MMP-3, MMP-9,MMP-13 and TIMPs)
Soluble receptor for advanced glycation endproducts (sRAGE)
YKL-40 (cartilage glycoprotein 39)

Osteoblast-Osteoclast Regulating Factors (Regulatory Molecules of Osteoblasts and Osteoclasts
Dickkopfs (Dkk-1)
Receptor activator of nuclear factor-kappaB ligand (RANKL)
Soluble Frizzled-related proteins (sFRP)
Sclerostin (SOST gene)
Wnt inhibitory factor-1 (WIF1)

Biomarkers Related to Other Metabolic Processes of Bone and Joint Tissues
Actin aortic smooth muscle
Adipokines (adiponectin, leptin, visfatin)
Aminopeptidase N (ANPEP)
Beta-2-microglobulin (B2M)
Biglycan
Brain and acute leukemia, cytoplasmic (BAALC)
Circulating microRNAs (miRNA 21, miRNA 23a, miRNA 24, miRNA 25, miRNA 100, miRNA 125b, miRNA 133a, miRNA148a, miRNA214, miRNA 503)
Cytokine-like 1 protein (CYTL1)
Dystrophin (DMD)
Fibronectin
Glycerophospholipid species (lyso)phosphatidic acid, (lyso)phosphatidylglycerol, and bis(monoacylglycero) phosphate, phosphocholine, phosphatidilcholine
Hemoglobin subunit alpha 2 or beta
Leukocyte cell-derived chemotaxin-2 (LECT2)
Metabolites (5-oxoproline, tyrosine, citric acid, lysine, acetylornithine, tryptophan, sarcosine, alanine and cisaconitic acid)
Oxylipins
Peroxiredoxin-6 (PRDX6)
Protein phosphatase 2A catalytic subunit (PPP2CA)
Signal transducer and activator of transcription 1 (STAB1)
Soluble receptor for leptin (sOB-Rb)
Sphingolipids (Sphingomyelins, ceramides and hexosylceramides and
dihexosylceramides)
Sphingosine 1-phosphate (S1P)
Tissue inhibitor of metalloproteinases-1 (TIMP1)
Tumori necrosis factor-alpha (TNF-α)-stimulated gene-6 (TSG-6)

### BRIEF DESCRIPTION OF THE DRAWINGS

FIG. 1. Histogram showing distribution of optical density values of SLP test results using in vitro model of infected synovial fluid sample with serial dilution of E. coli. Y axis represents optic density at minute 20 of the reaction.

FIG. 2. Histogram showing distribution of optical density values of SLP test results using in vitro model of infected synovial fluid sample with serial dilution of S. aureus. Y-axis represents optical density of the wells at minute 30 of the reaction.

FIG. 3. The graph at the top shows standard dose-response curves using serial dilution of peptidoglycan antigen from S. aureus (SA PG-Ag). X and Y axes represent time of reaction and optical density, respectively. A threshold of 15% change in light absorbance of the solution sample was determined and based on this threshold the concentration of the Staphylococcus aureus PG levels can be predicted by the time required to the occurrence of the threshold reaction (i.e. 15% change in the light absorbance) of the solution sample (depicted in the graph at the bottom).

FIG. 4. Standard dose-response curves using similar methodology as in FIG. 3, yet in a separate experiment in a different date, showing consistency of the positive control test and methodology applied for the experiments.

FIG. 5. SLP assay performed on non-infected synovial fluid samples.

FIG. 6. SLP assay performed on synovial fluid sample of patient #1. The patient was 66 year-old male who underwent revision knee surgery due to progressive pain over a course of more than one year following primary total knee replacement. This patient underwent revision surgery with preoperative diagnosis of aseptic loosening since most of the synovial and blood tests were negative for infection. He had positive culture of intraoperative periprosthetic tissue samples. The responsible pathogen in this case was Streptococcus intermedius.

FIG. 7. SLP test performed on synovial fluid sample from patient #2. The patient was a 64-year old female with early postoperative PJI of the right knee following primary total knee replacement. The responsible pathogen, identified by microbiologic culture of the periprosthetic tissue samples, was methicillin sensitive S. aureus.

FIG. 8-a. SLP test performed on synovial fluid sample from patient #3. The patient was a 78-year old male with past history of PJI of the right knee who underwent multiple revision surgeries including full course of two-stage exchange arthroplasty. The patient underwent revision surgery consisting of removal of the recently implanted prosthesis and implantation of an antibiotic releasing cement spacer. The responsible pathogen, identified by microbiologic culture of the periprosthetic tissue samples, was Candida tropicalis.

FIG. 8-b. SLP test performed on solid tissue sample from patient #3. The sample was taken during his recent surgery that consisted of exchange of antibiotic releasing spacer. The sample was obtained from tissue located between the bone and the prosthesis (interface tissue).

FIG. 9. SLP test performed on synovial fluid sample from patient #4. The patient was a 70-year old female with early postoperative PJI following left total hip replacement because of primary osteoarthritis. Two weeks following this intervention, he presented with severe pain and underwent revision surgery consisting of removal of the recently implanted prosthesis and implantation of an antibiotic releasing cement spacer. The responsible pathogen, identified by microbiologic culture of the periprosthetic tissue samples, was S. aureus.

FIG. 10. SLP test performed on synovial fluid sample from patient #5. The patient was a 54-year old male with multiple previous surgeries in his left hip due to PJI. He recently underwent revision surgery consisting of removal of antibiotic releasing cement spacer and implantation of his definitive prosthesis as the second stage of two-stage revision arthroplasty. SLP tests on his synovial fluid revealed low level of peptidoglycan (compare with standard dose-response curves in FIGS. 3 and 4), which seemed to indicate control of infection and therefore confirming the fact that the implantation of his definitive prosthesis was possibly placed in an appropriate time. The responsible pathogen, identified by microbiologic culture of the periprosthetic tissue samples from his previous surgery, was coagulase negative Staphylococcus, although samples of his re-implantation surgery were negative for culture and positive for SLP.

FIG. 11-a. SLP test performed on synovial fluid sample from patient #6. The patient was a 68-year old male with multiple previous surgeries in his right knee due to PJI. He recently underwent revision surgery consisting of removal of antibiotic releasing cement spacer and implantation of his definitive prosthesis as the second stage of two-stage revision arthroplasty. SLP tests on his synovial fluid revealed considerable levels of peptidoglycan (compare with standard dose-response curves in FIGS. 3 and 4), although preoperative blood and synovial fluid assays were negative. The responsible pathogen could not be isolated in this recent surgery but microbiologic cultures that were performed in the previous surgeries isolated methicillin sensitive S. aureus.

FIG. 11-b. SLP test performed on blood sample from patient #6. The blood was taken just before his recent surgery that consisted of removal of antibiotic releasing spacer and implantation of the definitive prosthesis.

FIG. 12. SLP test performed on synovial fluid sample from patient #7. The patient was a 69-year old male. He recently underwent revision surgery of his infected total right knee prosthesis consisting of removal of antibiotic releasing cement spacer and implantation of his definitive prosthesis as the second stage of two-stage revision arthroplasty. SLP tests on his synovial fluid was positive at two dilutions of 1:10 and 1:100 in a consistent manner (compare with standard dose-response curves in FIGS. 3 and 4), although preoperative blood and synovial fluid assays were negative. The responsible pathogen could not be isolated in this recent surgery but microbiologic cultures that were performed in the past isolated coagulase negative Staphylococci.

FIG. 13-a. SLP test performed on synovial fluid sample from patient #8. The patient was a 58-year old male with early postoperative PJI of the right knee following primary total knee replacement. Four weeks after his primary surgery, the patient underwent revision surgery consisting of removal of the prosthesis and implantation of an antibiotic releasing spacer. Four months later the patient underwent another revision surgery that consisted of exchanging his old spacer into a new one, because during the operation, the periprosthetic tissue did not have healthy appearance and the infectious process did not seem to be controlled. Furthermore, an intraoperative test of the synovial fluid (leucocyte esterase) was positive. The sample was taken during this last surgery. The responsible pathogen could not be isolated in his last surgery but previous microbiologic cultures had of the periprosthetic tissue samples taken in past surgeries isolated methicillin sensitive S. aureus.

FIG. 13-b. SLP test performed on blood sample from patient #8. The blood was taken just before his recent surgery that consisted of exchange of antibiotic releasing spacer.

FIG. 13-c. SLP test performed on solid tissue sample from patient #8. The sample was taken during his recent surgery that consisted of exchange of antibiotic releasing spacer. The sample was obtained from tissue located between the bone and the prosthesis (interface tissue).

FIG. 14-a. SLP test performed on frozen synovial fluid sample of patient #1

FIG. 14-b. SLP test performed on frozen synovial fluid sample of patient #2

FIG. 14-c. SLP test performed on frozen synovial fluid sample of patient #4

FIG. 14-d. SLP test performed on frozen synovial fluid sample of patient #5

### DESCRIPTION OF THE PREFERRED EMBODIMENTS

The preferred embodiments are best described by the following examples which are provided in support of the invention.

### Example 1

### Feasibility of PPO Test Using an In Vitro Model of Infected Synovial Fluid

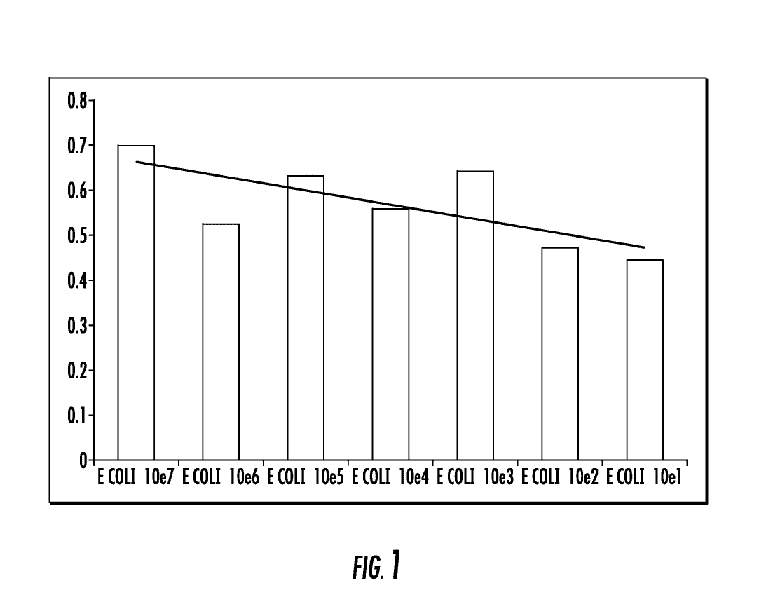

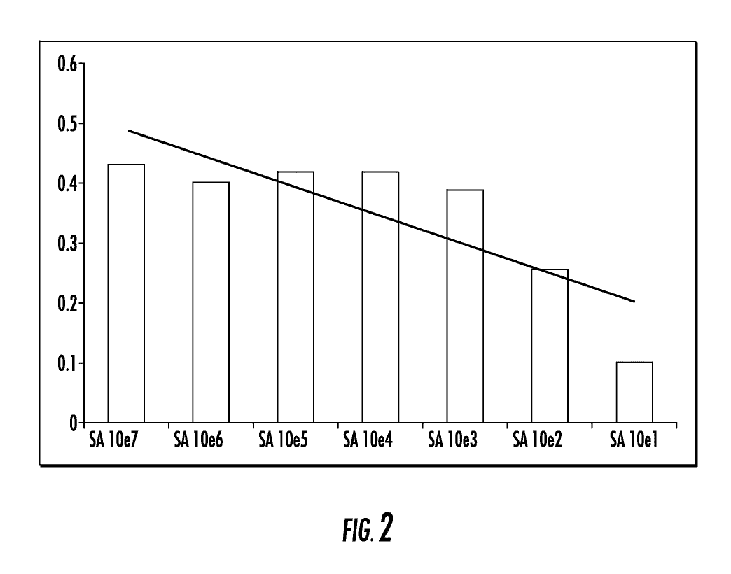

To assess feasibility and sensibility of the assay, in vitro models of infected synovial fluid samples were developed using Staphylococcus aureus (S. aureus) type ATCC 25923 and Escherchia coli (E. coli) type ATCC 25922. Non-infected synovial fluid samples were obtained from patients undergoing primary total hip or knee replacement in whom the procedure was performed to treat advanced joint osteoarthritis without any past history of infection in the joint. Bacteria were cultured in 10 ml of Trypticase Soy Broth (Becton Dickinson) in a shaker incubator (New Brunswick Scientific Inc, Edison, N.J.) overnight at 37° C. The following morning, bacteria were washed using sterile phosphate buffer saline (PBS) solution and centrifugation (14000 rpm for 5 min). Bacteria were added to sterile PBS solution. Serial 100 ul amounts of whirled bacterial solution were added to 900 ul of sterile PBS until turbidity meter (Dave Barry's) read the sample as 0.10-0.11 that was equivalent to 10⁸ colony-forming units per milliliter (CFU/ml). Serial dilution was performed 7 times to achieve solutions containing 10⁷ to 10¹ CFU/ml of either S. aureus type ATCC 25923 or E. coli type ATCC 25922 in synovial fluid. Petri films (3M) were incubated with 1 ml solution containing 10² to 10⁴ CFU/ml and were incubated overnight at 37° C. and visible colonies were counted next day to confirm CFU counts of the original solution. 50 ul of each solution was added to 50 ul of silkworm larvae plasma (SLP) reagent solution in microplate wells as instructed by the developer (WAKO chemicals USA, Richmond). The microplate was placed in a microplate reader using a colorimetric assay by measuring light transmittance of the solution of the wells. The setting for microplate reader included wavelength 650 nm, room temperature and 180 cycles of reading with 30-second intervals. (FIGS. 1 and 2)

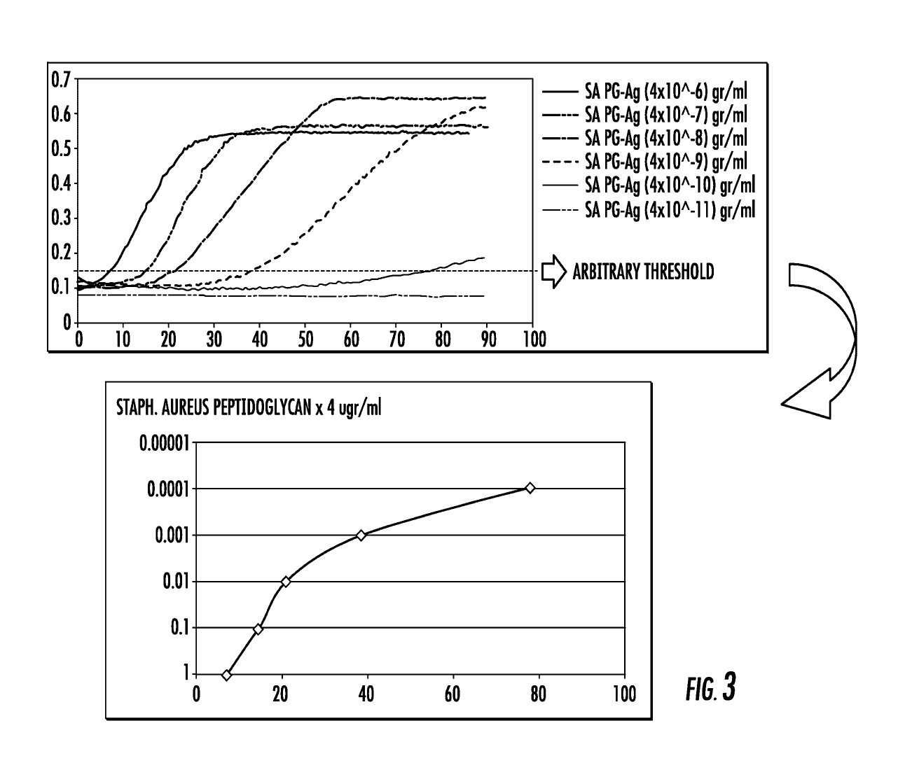

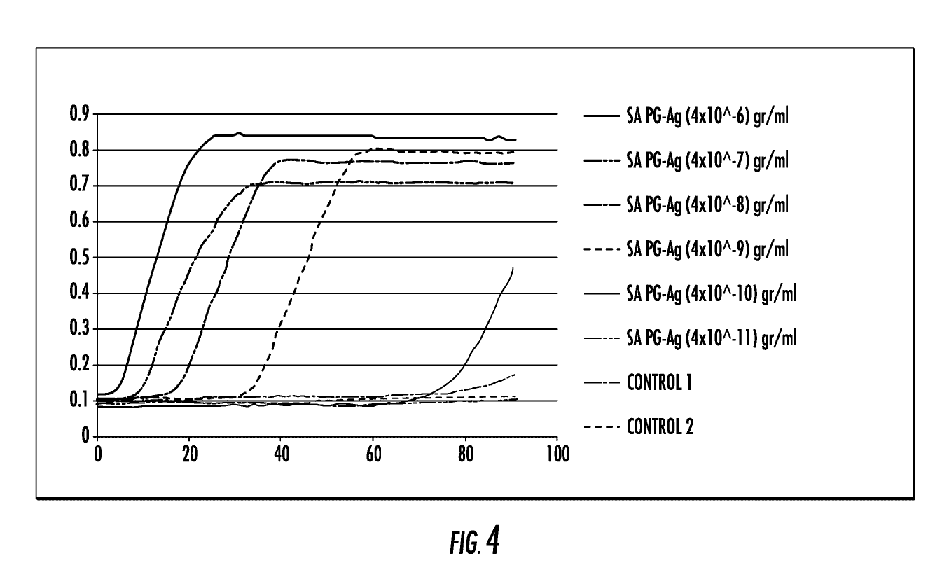

In order to obtain standard control graphs (positive controls), four serial 10-fold dilutions were performed on the solution containing 4 ugr/ml of peptidoglycan (PG) antigen of S. aureus (provided by WAKO chemicals USA Richmond) to obtain dilution of 40 pg/ml of S. aureus PG antigen. Dilutions were performed using water for injection. (FIGS. 3 and 4)

Several tests were conducted as negative control experiments. One series of control experiments consisted of two 50-ul samples from water for injection (used for dilution of synovial fluid, synovial tissue and blood samples). The experiment with negative control samples (water with injection used for dilution) was repeated in each set of experiments with in vitro models or real clinical samples.

### Example 2

### Experiments for Evaluating PPO Test on Clinical Samples from Non-Infected Synovial Fluid Samples

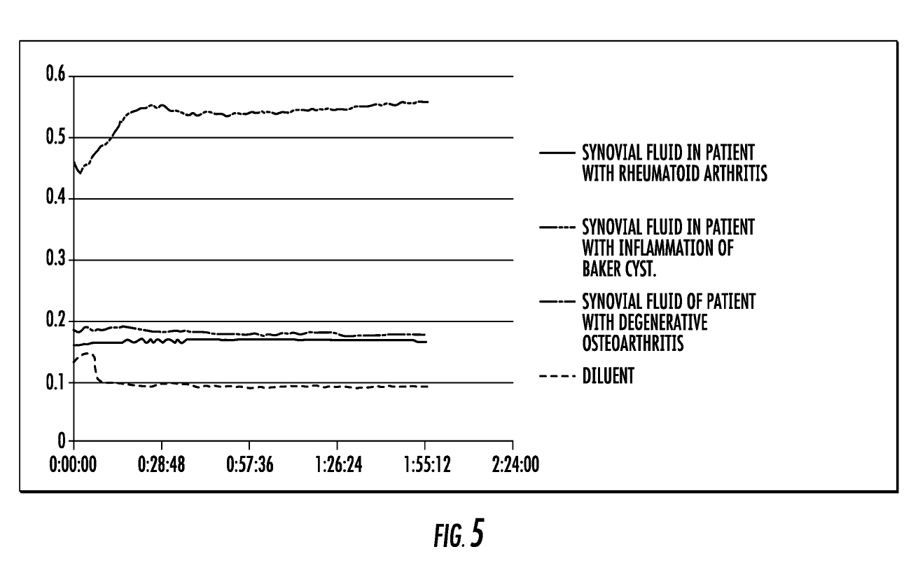

In another series of experiments synovial fluid samples from three different patients with non-infected knee joints including one with primary degenerative osteoarthritis, one with rheumatoid arthritis and one with inflamed Baker's cyst were tested with SLP reagent in undiluted state and dilution of 1:50 using water for injection. The water used for dilution was also tested. All negative controls were assayed using SLP reagent in the same microplate wells and using similar microplate reader setting as described earlier (FIG. 5).

### Example 3

### Feasibility of the PPO Test for Real Clinical Samples

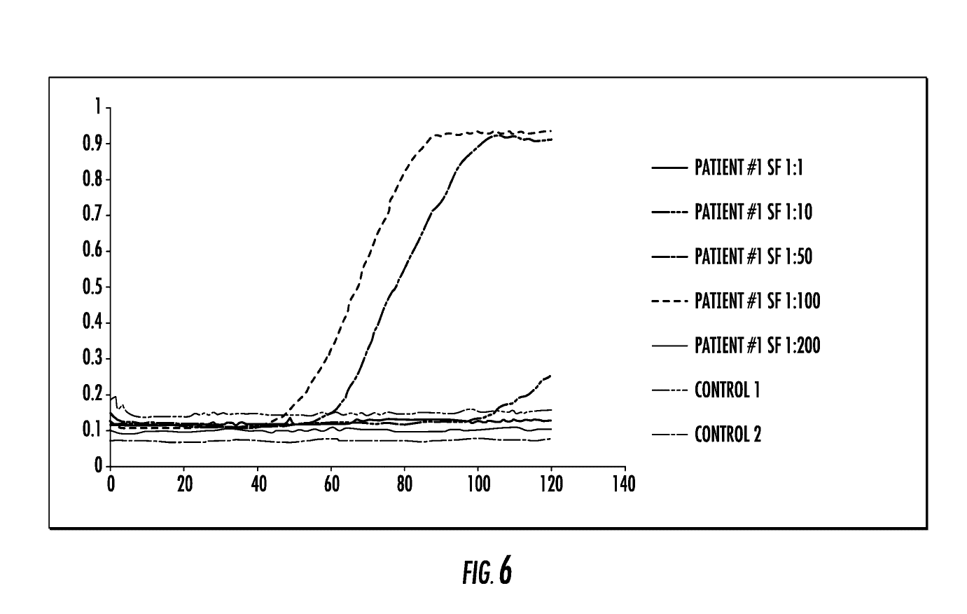

Following the preliminary experiments with in vitro models and negative controls, several experiments were conducted using samples from preoperative aspiration or intraoperative sampling of synovial fluid from prosthetic knee or hip joints that underwent revision surgery because of already confirmed or suspected prosthetic infection. SLP assay was performed for these clinical synovial fluid samples in several dilutions including 1:1 (undiluted), 1:10, 1:50, 1:100 and 1:200 using water for injection for dilution. In one clinical scenario, a 66-year old male patient (hereby named as patient #1) presented with progressive right knee pain. As past surgical history, patient #1 had undergone primary total knee replacement due to osteoarthritis two years ago. The patient had complications regarding his surgical wound healing that took several weeks to heal. However, he did not have any sign of deep infection. Shortly after recovery from right total knee replacement, he started to notice progressive pain and effusion in his knee. The patient was evaluated in our institution for pain in his prosthetic right knee. Preoperative evaluation including blood markers for infection (erythrocyte sedimentation rate and C-reactive protein) and aspiration of periprosthetic fluid were negative for infection. However, tri-phasic bone scan suggested an infectious process. Revision knee arthroplasty with an impression of presumably aseptic loosening of his components was recommended. The patient underwent revision surgery and following the surgery, the results of culture of intraoperative sample of periprosthetic tissue was reported to be positive for Streptococcus intermedius. Considering history of surgical wound healing complications at the time of primary knee joint arthroplasty, onset of progressive pain shortly after the index surgery and preoperative tri-phasic bone scan being positive, the surgeon decided to treat the patient as a periprosthetic joint infection (PJI) and the patient started oral antibiotic therapy afterwards. The SLP test was performed on intraoperative samples of this patient and was observed to be positive at dilutions of 1:50 and 1:100. Two negative controls were also performed during this assay (FIG. 6)

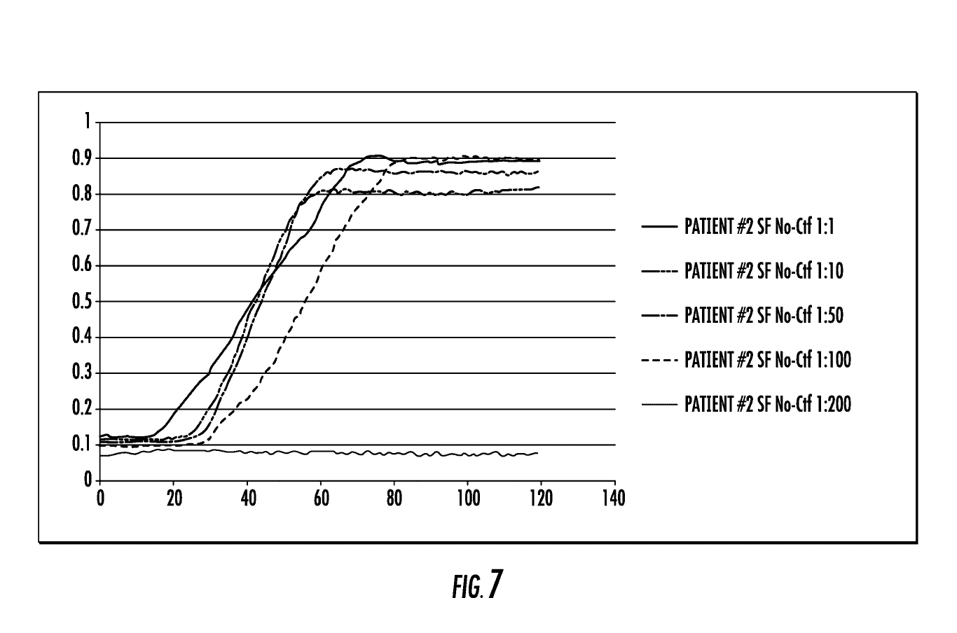

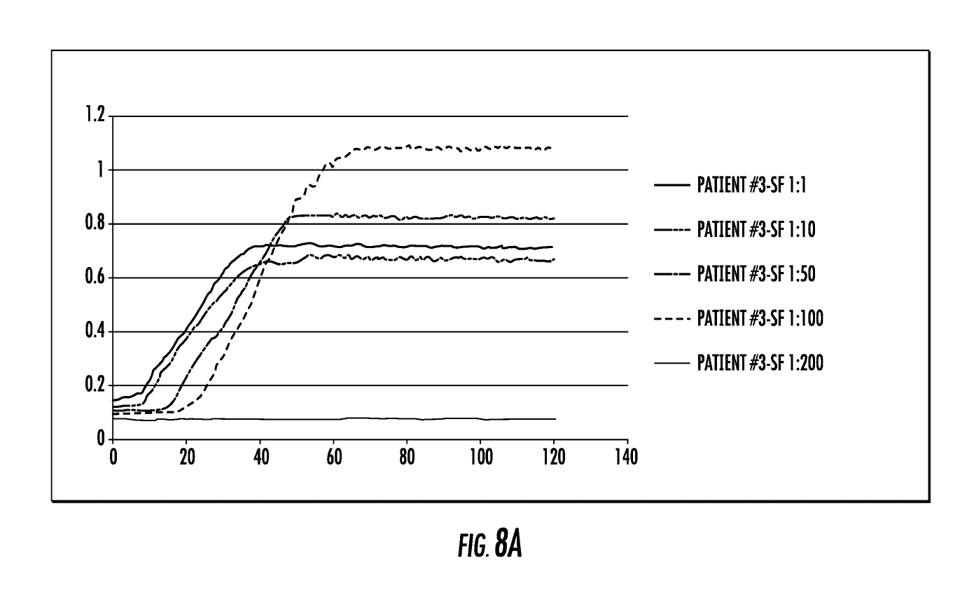

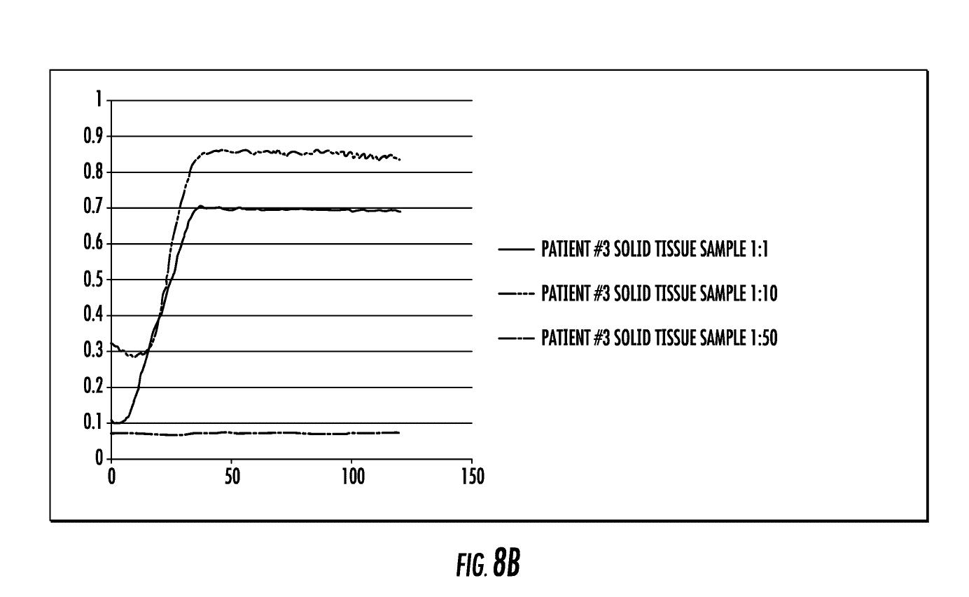

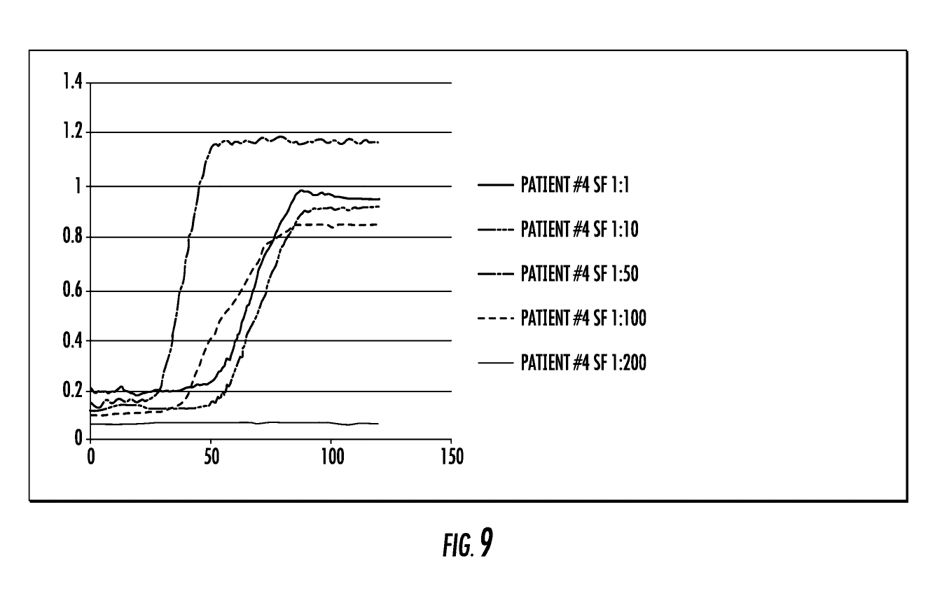

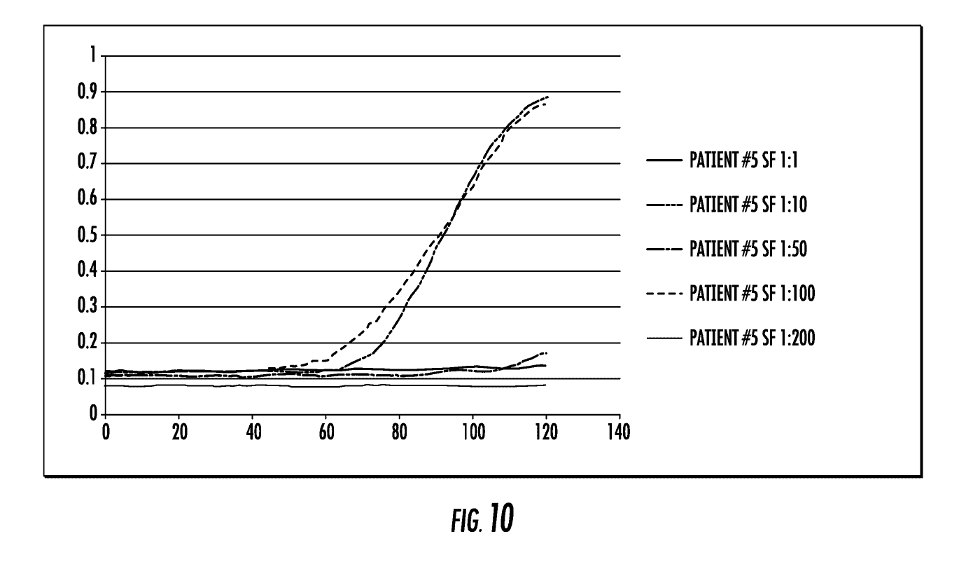

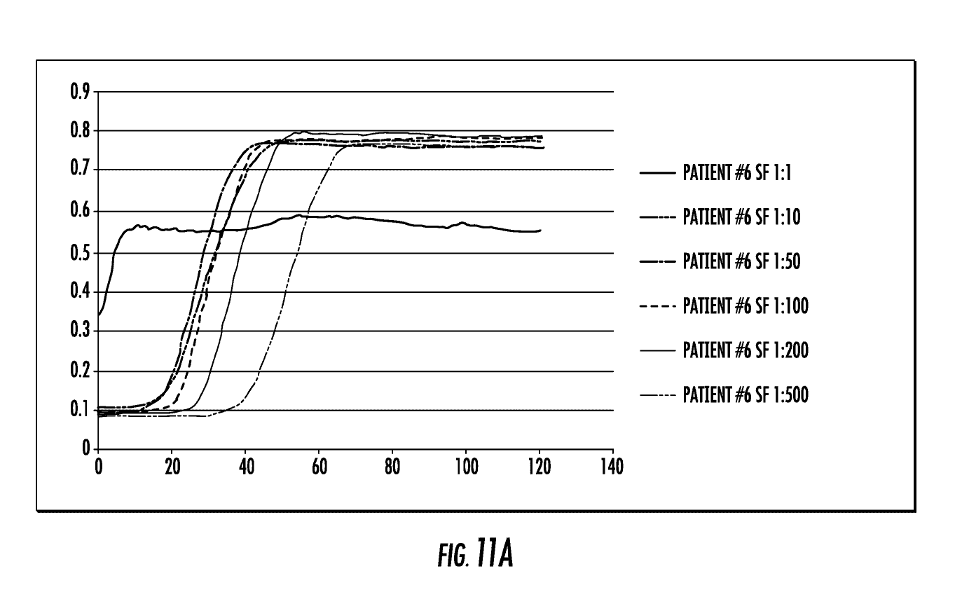

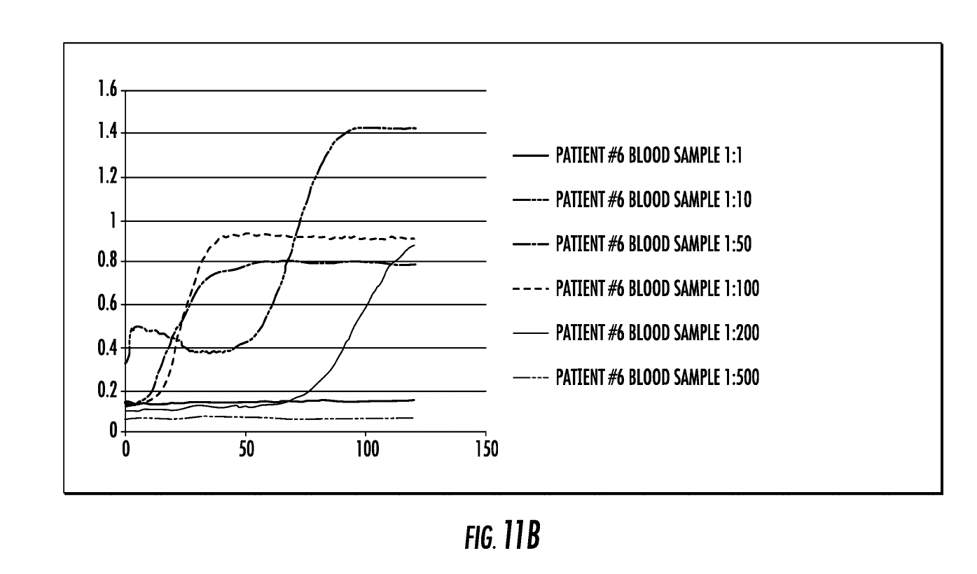

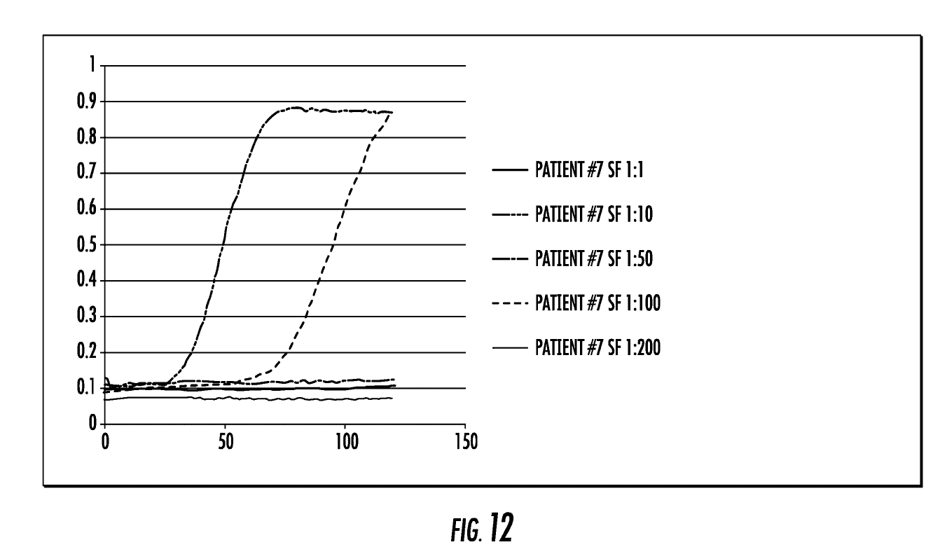

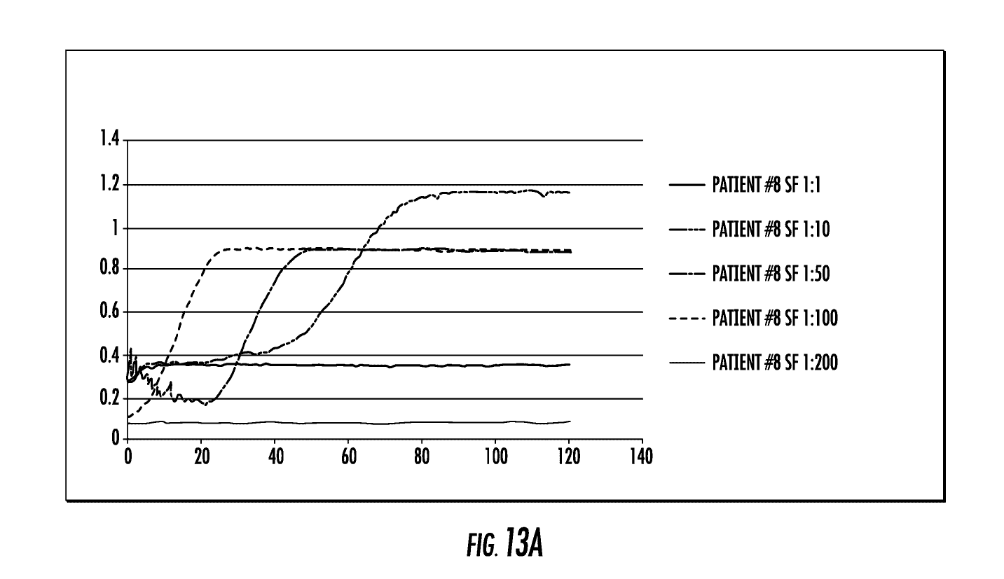

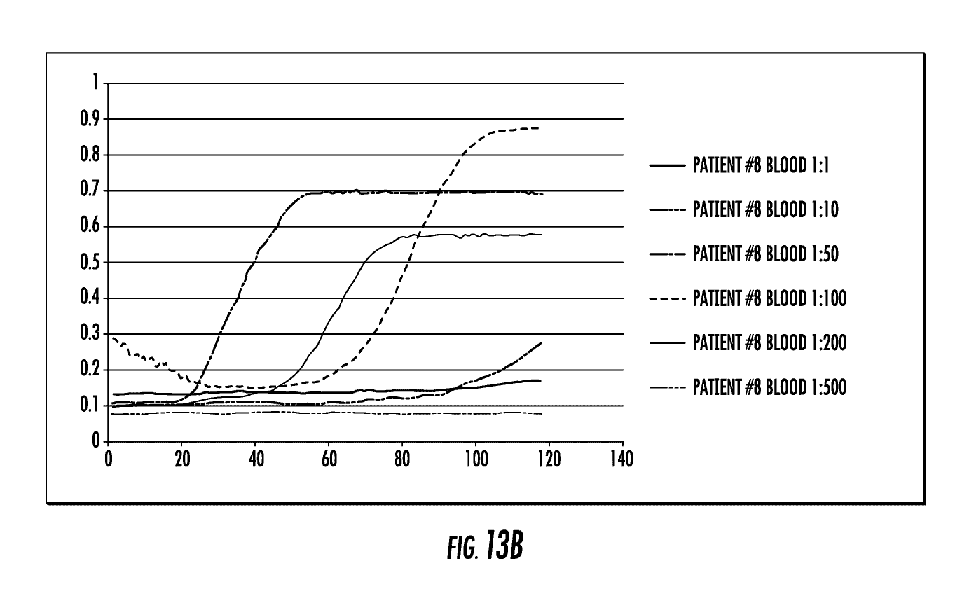

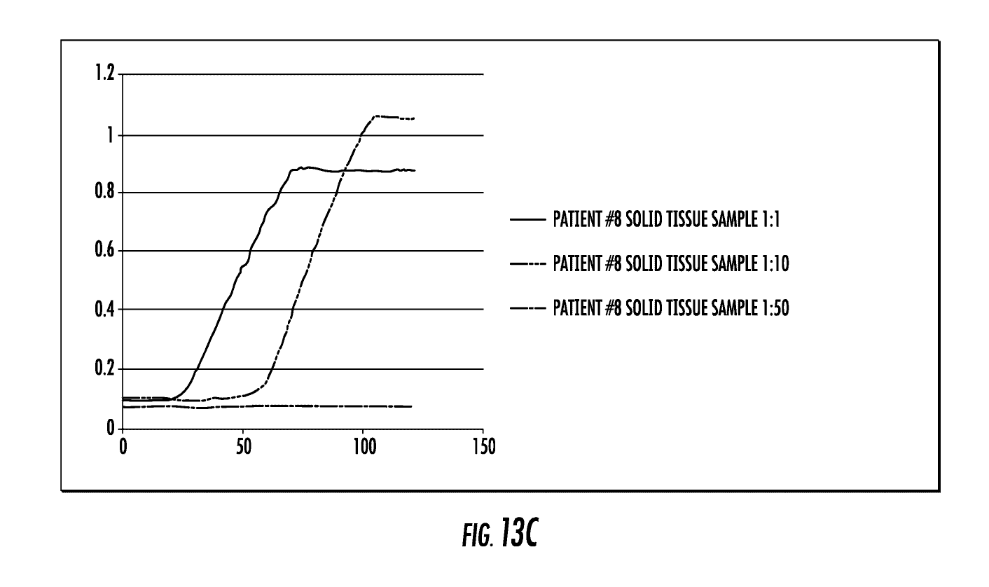

The infected synovial fluid samples were collected from patients with periprosthetic joint infection in different stages of the evolution of the disease and its treatment. Examples were cases with early postoperative periprosthetic joint infection (patient #2, [FIG. 7], patient #4 [FIG. 9]), cases with recurrence of periprosthetic joint infection (patient #3 [FIG. 8], patient #5 [FIG. 10], patient #6 [FIGS. 11-a and 11-b]) and patients in the treatment process of an already diagnosed periprosthetic join infection who underwent several surgical procedures in the past (patient #3 [FIG. 8], patient #5 [FIG. 10], patient #6 [FIGS. 11-a and 11-b], patient #7 [FIG. 12]). These surgical procedures included removal of implant and placement of antibiotic-laden cement spacer (patient #2 [FIG. 7], patient #3 [FIG. 8]), exchange of antibiotic spacer into another spacer (patient #8 [FIG. 13]), and removal of antibiotic spacer and implantation of definitive prosthesis (patient #5 [FIG. 10], patient #6 [FIGS. 11-a and 11-b], patient #7 [FIG. 12]). Blood samples were also obtained from patients undergoing the second stage of two-stage exchange arthroplasty (removal of antibiotic spacer and implantation of definitive prosthesis) due to a confirmed periprosthetic joint infection. Blood samples were withdrawn just prior to start of surgery (patient #9, FIG. 13-b). Tissue samples were obtained during surgical procedures. Solid tissues originated from the joint capsule or from prosthesis-bone interface (located between the prosthesis and the bone). Solid tissue samples were cut into small pieces in a sterile petri dish plate and were placed in Eppendorf tubes. Water for injection was added to Eppendorf tubes containing tissue samples and the tubes were whirled for 2 minutes. The water after whirling was utilized for testing the peptidoglycan or beta-glucan measurement (patient #8, FIG. 13-c). In each series of testing of clinical samples, a negative control experiment was included in the same microplate under the same test conditions. Water for injection used for dilution of synovial fluid samples or for obtaining periprosthetic tissue broth was used as negative control.

### Example 4

### Influence of Freezing of Infected Synovial Fluid Samples on the Result of PPO Test

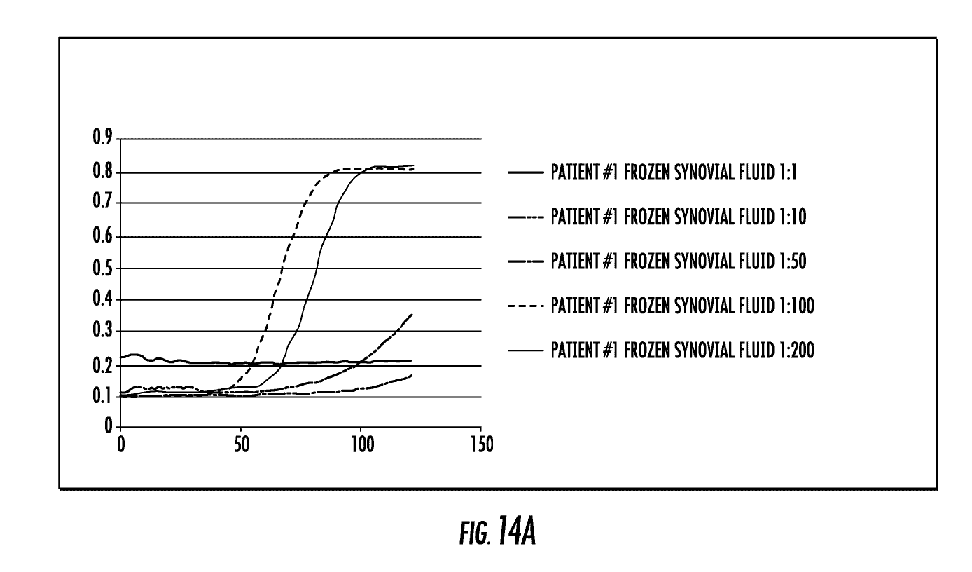

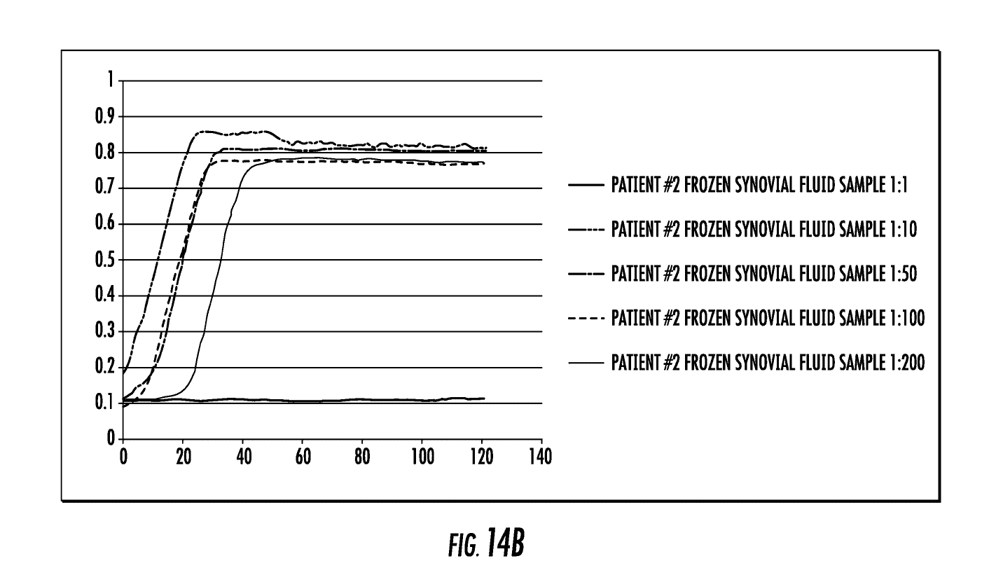

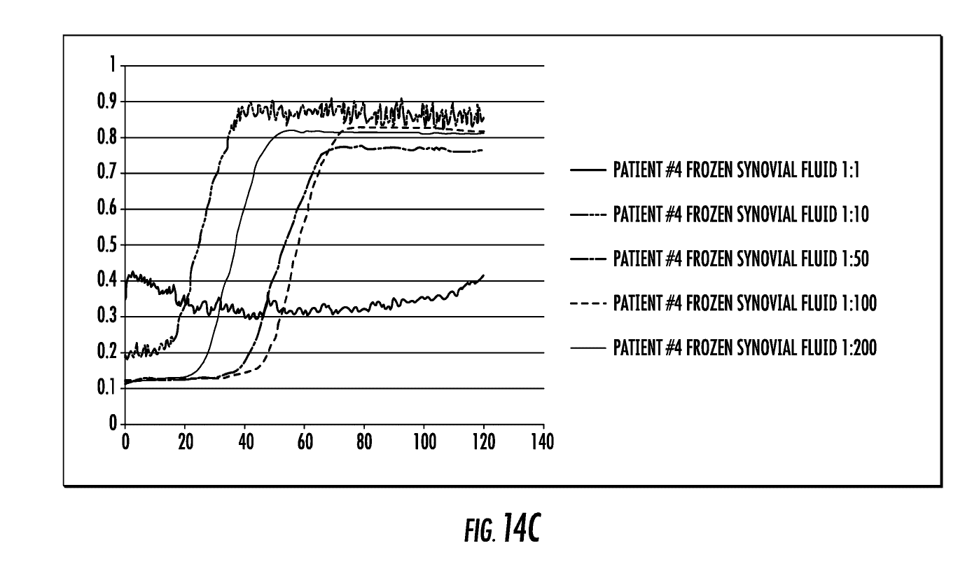

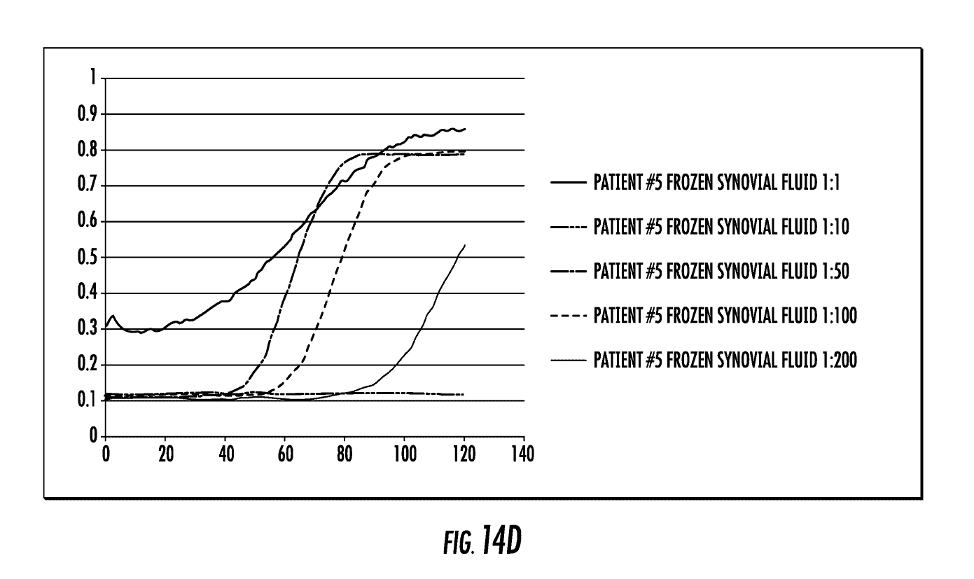

Four samples of infected synovial fluid (from patients #1,2,4 and 5) were assessed by SLP test before and after 7-10 days of storage at −20° C. Graphs are presented in FIGS. 14-a, 14-b, 14-c and 14-d.

### Example 5

### Measurement of Biomarkers of Bone Destruction in the Blood of Patients with Periprosthetic Joint Infection

Out of 9 blood samples of patients with periprosthetic joint infection that were tested for different biomarkers, in five samples the levels of osteopontin were found to be higher than the reference normal range of (7.2-40 ng/ml). The values that were detected were 44, 51, 55, 64, 88 and 219 ng/ml. In three samples from the same group the levels of osteoprotegerin were higher than reference range of 2.3-8.4 pM and were detected to be 9.6, 10 and 11 pM. Two other patients had levels very close to the upper normal limit of the reference range with the values being 8.2 and 8.3 pM. The elevated level of biomarkers indicative of bone destruction together with the elevated level of SLP is likely to be specific for periprsthetic joint infection. The combination of the biomarker indicative of bone destruction together with SLP allows us to reduce the incidence of false positive SLP tests that may be seen in patients with infection arising from sources other than the joint.

Modifications and variations of the foregoing will be apparent to those having skill in the art based upon the disclosures provided herein. For example, variations on dilution, use of centrifuges and use of enzymes (such as collagenase, plasmin, etc.) to overcome clumps and improve the quality of interface tissue material.

1. A system for use as a diagnostic assay for the diagnosis of peri-implant infections of bone and joints in a subject with pen-implant infection of bone and joints comprising
at least two separate tests in blood, urine, synovial fluid and/or pen-implant tissues obtained from the subject wherein detection of peptidoglycan or beta-glucan is made by one test and detection of biomarkers of destruction of bone, cartilage, muscle, synovial, fat and other pen-implant tissues is made by another test.

2. A test sample for use in a diagnostic assay for detection of peptidoglycan or beta-glucan comprising
extract of hemolymph of an insect having prophenoloxidase activity against peptidoglycan or beta-glucan with end product of melanin and a sample from a patient comprising diluted blood, urine, synovial fluid or liquefied tissue.

3. The test sample of claim 1 wherein the insect is a silk worm.

4. The test sample of claim 2 wherein the hemolymph is obtained from Bombyx mori larvae.

5. The test sample of claim 1 wherein the dilution is from undiluted to about 1:500 or about 1:200.

6. (canceled)

7. The test sample of claim 1 wherein the hemolymph is lyophilized.

8. A diagnostic assay method for detection of peptidoglycan or beta-glucan in samples of blood, urine, synovial fluid or samples of solid pen-implant tissues of patients with possible peri-implant infection of bone and joints comprising
making liquid test samples by serial dilution of blood, urine and synovial fluid samples or liquid tissue samples obtained from vibration of solid tissue samples in highly purified water, followed by
addition to the test samples of extract of hemolymph of an insect (such as silk worm) with prophenoloxidase activity against peptidoglycan or beta-glucan with end product of melanin, and then
performing a colorimetric assay in which darkness of the solution determines the presence of peptidoglycan or beta-glucan and therefore confirmation of the presence of bacterial or fungal periprosthetic joint infection.

9. The method of claim 8 in which the sample is selected from the group consisting of blood, urine, synovial fluid, periprosthetic fluid or any liquid or solid tissue around prosthetic implants placed in physiologically sterile anatomic sites.

10. The method of claim 8 in which the fluid sample is diluted in several serial dilutions from undiluted up to 1:500 or 1:200.

11. (canceled)

12. The method of claim 8 in which the solid tissue is cut into small pieces and vibrated in sterile highly purified water to obtain liquid sample.

13. The method of claim 8 in which the sample is placed in contact with a natural solution with prophenoloxidase pathway activity.

14. The method of claim 8 in which the natural solution with prophenoloxidase pathway is hemolymph obtained from Bombyx mori (silk worm) larvae.

15. The method of claim 8 in which hemolymph is lyophilized.

16. The method of claim 8 in which the test will take up to about 120 minutes to complete.

17. The method of claim 8 in which colorimetric assay using microplate reader is used to assess the change of light transmittance or light absorbance of the solution at a wavelength of 650 nm.

18. A method of diagnosing infection in a subject consisting of detecting the presence of a biomarker of peri-implant tissue degradation in blood, urine, synovial fluid or liquefied solid samples from the pen-implant tissues.

19. The method of claim 18 wherein the biomarker is selected from the group consisting of biomarkers of bone metabolism, degradation or remodeling, Biomarkers related to collagen metabolism, Biomarkers related to aggrecan metabolism, Biomarkers related to other non-collagenous proteins, Osteoblast-osteoclast regulating factors (Regulatory molecules of osteoblasts and osteoclasts and Biomarkers related to other metabolic processes of bone and joint tissues (Table) and any combination thereof.

20. The method of claim 19 wherein the biomarker can be measured by an antibody-based immunoassay method or other non-immunoassay methods.

21. The method of claim 20 wherein the antibody-based immunoassay method is selected from the group consisting of enzyme-linked immunosorbent assay or ELISA, radioimmunoassay or RIA, and lateral flow immunoassay.

22. The method of claim 20 wherein the non-immunoassay methods are proteomic methods.
